(19) United States
(12) Patent Application Publication
(10) Pub. No.: US 2024/0174980 A1
(43) Pub. Date: May 30, 2024

(54) PROCESS FOR MANUFACTURE OF A COMPOSITION FOR THE TREATMENT OF OSTEOARTHRITIS IN A SUBJECT

(71) Applicant: STEM CELLX EUROPE KFT, Szeged (HU)

(72) Inventors: Éva KRISTON-PÁL, Szeged (HU); Valéria SZUKACSOV, Szeged (HU)

(73) Assignee: CELLFACTORY KFT., Szeged (HU)

(21) Appl. No.: 18/284,831

(22) PCT Filed: Mar. 29, 2022
(86) PCT No.: PCT/HU2022/050027

§ 371 Date: Sep. 28, 2023

(30) Foreign Application Priority Data
Apr. 1, 2021 (HU) P2100138

(51) Int. Cl. C12N 5/0775; A61K 35/28; A61P 19/02

(52) U.S. Cl. CPC C12N 5/0667; A61K 35/28; A61P 19/02; C12N 2500/62; C12N 2501/20

(57) ABSTRACT

The method for the manufacture of a composition for the treatment of osteoarthritis in subjects involves the use of mesenchymal stem cells (MSC). These MSCs are derived from at least two healthy donors who are different from the subject to be treated. The MSCs are morphologically healthy and substantially free of senescent cells. They are also free of pathogens characteristic of the species and possess the main characteristics of MSCs. During propagation, the cumulative population doubling value (CPD value) of the MSCs is preferably in the range of 3 to 14. The level of kynurenine metabolite production of the MSCs is in the range of 30 to 50 ng/ml, and the level of THBS1 cytokine production of the MSCs is in the range of 50 to 80 ng/ml. The number of cells used is in the range of 105 to 5×107.

### TECHNICAL FIELD

The present invention relates to a process for the manufacture of a composition for the treatment of osteoarthritis in subject, in particular mesenchymal stem cells isolated, and propagated in a specific way, and use thereof.

### BACKGROUND ART

Articular inflammation—also called osteoarthritis—is a disease that can occur both in humans and animals at any age. It is most prevalent at older age (generally after the age of 7 years in dogs), due to articular cartilage wear and the gradual weakening of the ligaments.

The EP2504426 (B1) (STEMPEUTICS RES PVT LTD [IN]) relates to a process for the isolation, collection, and further propagation of mesenchymal stem cells (MSCs) that can be used for clinical applications. The process relates to the production of a composition comprising mesenchymal stem cells, Plasmalyte A, human serum albumin (HSA), dimethyl sulfoxide (DMSO) and, optionally pharmaceutically acceptable additives. The essence of the process is the propagation of the MSCs to an appropriate amount to prepare products for clinical application. The document describes the propagation of human MSCs derived from bone marrow. The markers affecting the various differentiation abilities of the MSCs did not influence the selection during propagation. The cells were not screened for pathogens during MSC propagation.

According to the process described in EP1276486 (B8) specification, it comprises the administration of MSCs into the joint for the regeneration of the cartilage tissue in the joint. In the description, use of allogeneic MSCs is disclosed for the production of a pharmaceutical composition for the improvement of animal joints. The composition is used to treat the damaged joint of the animal and to improve joint tissue. It describes the general use of MSCs, with the MSCs derived from goat bone marrow, and apart from propagation, the MSCs were not subjected to any specific, marker-based selection.

In the P1500218 Hungarian patent application, a composition is described for the treatment of joint damage, preferably joint dysplasia in individuals of the given mammalian species. The preparation comprises allogeneic MSCs produced by a procedure involving isolation and propagation from donor individuals preferably diagnosed to be free of dysplasia and in the given case known diseases, as well as anionic, non-sulphated glycosaminoglycan, preferably hyaluronic acid at a concentration of 0.1-1 wt %. The MSCs are derived from the visceral adipose tissue of the donors. During the propagation of the MSCs, the cells were not screened for pathogens. The MSCs were not subjected to any specific, marker-based selection during propagation. Furthermore, the use of hyaluronic acid in intraarticular injections for the amelioration of the symptoms of osteoarthritis (OA) is not authorized for every species, thus, we aimed at achieving an MSC product that does not contain hyaluronic acid.

Annalisa Guercio et al. [Annalisa Guercio et al., Cell Biology International, 36(2), (2012), 189-194] describe the use of MSCs derived from the dog's own adipose tissue in dogs suffering from chronic inflammation of the humeroradial joints. The publication thus describes an autologous AD-MSC therapy, during which the necessary stem cells are derived from the subject to be treated and then re-administered to the subject. Before re-administration, the cells are screened for microbiological contamination, differentiation ability, and gene expression markers. However, the document does not mention any selection steps that would be based on specific trombospondin-1 cytokine (THBS1) production or kynurenine metabolite production.

Kriston-Pál et al. [Kriston-Pál et al., The Canadian Journal of Veterinary Research, 2017, 81; 73-78] describe AD-MSCs that had been selected for therapeutic purposes. The aim of the study was to examine the effect of a hyaluronic acid solution of MSCs on the regeneration of elbow joints with articular inflammation in dogs. The study docs not describe the relationship between the regenerative effects of MSCs and the specific levels of the metabolite kynurenine and/or the cytokine thrombospondin-1. The use of hyaluronic acid is not permitted in certain individuals; thus, there is a need for the development of a composition that does not contain hyaluronic acid but shows good result regarding efficacy.

Maumus et al. [Maumus et al., Frontiers in Immunology, 2017 (8), 1638] aimed at examining, via secretome analysis, whether the THBS1 cytokine levels play a role in the cartilage protective and anti-inflammatory roles of ASC cells. Although the role of the THBS1 cytokine is studied extensively, the document does not mention either the suppression of the hypertrophic chondrocyte phenotype, which is a result of the decrease in the expression of GADD45 and CEBPb and the MMP13 protein they target, or the role of the production of the metabolite kynurenine.

The information disclosed in the above-mentioned documents of the prior art is incorporated herein by reference, with special regard to the description of the applied definitions and methods of propagation.

Osteoarthritis (articular inflammation) is a multifactorial disease, that is, not a single but several different factors contribute to its development. There is genetic predisposition to it, but it may also arise due to a previous trauma or injury. It may also be caused by developmental disorder of the bones comprising the joint (hip or elbow dysplasia) or abnormal wear of the articular cartilage. The risk of its development is elevated by obesity, which can exacerbate the condition by overloading the joints. It is generally the hip, knee, or elbow joint that is affected.

This disease is mostly, though not exclusively, of genetic origin, a hereditary disease and not necessarily the result of injury. Thus, in order to provide optimal treatment, this point should be considered as well, and in order to obtain MSCs of appropriate efficacy, it is important to ensure an allogeneic source.

A further factor in obtaining allogeneic MSCs is that no invasive intervention should be required for this purpose; tissues removed and handled as waste from individuals that have to be otherwise subjected to an elective intervention should be used.

It is generally known that diseases of this type can be treated with mesenchymal stem cells (MSC), but the efficacy of the treatment can vary.

The aim of the invention is to eliminate the shortcomings of previous solutions and to develop an MSC-containing composition for the treatment of a subject suffering from osteoarthritis that enables the regeneration of cartilage tissue in the subject suffering from osteoarthritis and thus recovery from the health problem manifested as lameness.

### SUMMARY OF INVENTION

### Technical Problem

There is thus a further need for a method and a composition with which osteoarthritis, that is, articular degeneration can be treated in a simple and efficient way using adipose tissue derived allogeneic MSCs that are efficient because their differentiation can be directed if certain conditions are fulfilled, which results in a more efficient realization of treating the illness. It is a further important aspect that the use of the allogeneic MSC-derived final product should be as safe as possible, which was not appropriately revealed in the documents described in the prior art technology.

### Advantageous Effects of Invention

Surprisingly, the inventors found that the AD-MSCs, which are obtained via a known method, propagated in several steps, and then selected, can efficaciously suppress the development of the hypertrophic chondrocyte phenotype at an appropriate concentration during the differentiation of the MSCs, a process which promotes the restoration of the articular tissue in abnormal conditions.

Surprisingly, the inventors found that those AD-MSCs that produce the metabolite kynurenine and the THBS1 cytokine in specific amounts can efficaciously suppress the development of the hypertrophic chondrocyte phenotype during the differentiation of the MSCs.

Reducing the development of the hypertrophic chondrocyte phenotype results in faster recovery since a larger proportion of the implanted MSCs may differentiate into healthy chondrocyte cells, leading to a quick and efficient restoration of the cartilage tissue.

Thus, our inventive finding is that specific levels of kynurenine and THBS1 production of MSCs present in the isolates used to treat osteoarthritis are responsible for the differentiation, responsible for the regenerative effect of MSCs.

Furthermore, the quality of the isolated MSCs is also determined by the cytokine profile they produce, and the doubling time (PD, Population Doubling) determined during propagation. The former ensures that the preparations of different productions are composed of MSCs of similar quality, while the latter enables the production of a safe, non-tumorigenic preparation by keeping the final population duplication (CPD, Cumulative Population Doubling) value below 12. The importance of controlling the CPD value lies in avoiding and filtering out tumour-like MSCs that would be prone to proliferation in a disadvantageous direction during their differentiation.

It also contributes to the safe use of the product that we test for the presence of 26 pathogens occurring in dogs, and only use verified pathogen-free MSCs for the final product.

### BRIEF DESCRIPTION OF DRAWINGS

The subject matter of the invention encompasses allogeneic MSCs that possess the following characteristics:
they are derived from at least two healthy donors that are different from the individual to be treated;
they are morphologically healthy, substantially free of senescent cells;
they are free of pathogens;
they possess the main characteristics of MSCs; they are CD105 and CD73 positive but do not carry surface markers characteristic of hematopoietic stem cells and progenitor cells and preferably do not carry surface markers characteristic of blood cell development lineages, that is, the MSCs are preferably CD45, CD34, CD14, CD11b, CD31, CD79α, and CD19 negative;
during propagation, the CPD value of the MSCs is preferably in the range of 3 to 14, more preferably 2 to 12, and most preferably about 8;
the level of kynurenine metabolite production of the MSCs is in the range of 30 to 50 ng/ml, preferably about 30 ng/ml, and the level of THBS1 cytokine production of the MSCs is in the range of 50 to 80 ng/ml, preferably about 50 ng/ml.

The subject matter of the invention further encompasses a composition that comprising of:
a) allogeneic MSCs that possess the following characteristics:
they are derived from at least two healthy donors that are different from the individual to be treated;
they are morphologically healthy, substantially free of senescent cells;
they are free of pathogens;
they possess the main characteristics of MSCs; they are CD105 and CD73 positive but do not carry surface markers characteristic of hematopoietic stem cells and progenitor cells and preferably do not carry surface markers characteristic of blood cell development lineages, that is, the MSCs are preferably CD45, CD34, CD14, CD11b, CD31, CD79α, and CD19 negative;
during propagation, the CPD value of the MSCs is preferably in the range of 3 to 14, more preferably in the range of 2 to 12, most preferably about 8;
the level of kynurenine metabolite production of the MSCs is in the range of 30 to 50 ng/ml, preferably about 30 ng/ml, and the level of THBS1 cytokine production of the MSCs is in the range of 50 to 80 ng/ml, preferably about 50 ng/ml; and
b) one or more pharmaceutically acceptable additives.

Preferably, the one or more pharmaceutically acceptable additive is a cryoprotective agent, preferably DMSO-containing Hank's balanced salt solution (HBSS).

Preferably, the number of allogeneic MSCs is in the range of 10⁵ to 10⁸, preferably in the range of 10⁵ to 5×10⁷ in the composition.

Preferably, the MSCs are derived from at least two separate, preferably 3 to 15, more preferably 4 to 8, most preferably 5 to 7 donors that belong to the same species.

Highly advantageously, the composition is a balanced preparation that contains the MSCs selected from the separate donors mixed in a way that fewer cells are dispensed into the end product from those isolated MSCs that produce more kynurenine and THBS1 cytokine and more from those MSCs that produce less kynurenine and THBS1 cytokine, thus, we obtain a balanced mixture of MSCs with similar total kynurenine and total THBS1 cytokine production in the different amplification cycles.

The object of the present invention further relates to allogeneic MSCs for the treatment of osteoarthritis in a subject in needs thereof, wherein allogeneic MSCs possess the following characteristics:
they are derived from at least two healthy donors that are different from the individual to be treated;
they are morphologically healthy, substantially free of senescent cells;
they are free of pathogens;
they possess the main characteristics of MSCs; they are CD105 and CD73 positive but do not carry surface markers characteristic of hematopoietic stem cells and progenitor cells and preferably do not carry surface markers characteristic of blood cell development lineages, that is, the MSCs are preferably CD45, CD34, CD14, CD11b, CD31, CD79α, and CD19 negative;
during propagation, the CPD value of the MSCs is preferably in the range of 3 to 14, more preferably in the range of 2 to 12, most preferably about 8;
the level of kynurenine metabolite production of the MSCs is in the range of 30 to 50 ng/ml, preferably about 30 ng/ml, and the level of THBS1 cytokine production of the MSCs is in the range of 50 to 80 ng/ml, preferably about 50 ng/ml.

The object of the present invention further relates to a composition comprising allogeneic MSCs for the treatment of osteoarthritis in a subject in needs thereof.

The MSCs are preferably derived from at least two separate, preferably 3 to 15, more preferably 4 to 8, most preferably 5 to 7 donors that belong to the same species.

Preferably, the MSCs are derived from stromal vascular fraction (SVF) which is isolated from abdominal adipose tissue removed during routine ovariohysterectomy.

Preferably, the number of MSCs used for the treatment of a single joint is in the range of 10⁵ to 10⁸, preferably in the range of 10⁵ to 5×10⁷.

Preferably, the MSCs or composition is administered intraarticularly or in the form of implantation.

According to the preferred embodiment, the subject is a non-human mammal. According to a more preferred embodiment, the subject is herbivorous, carnivorous, or omnivorous. According to the preferred embodiment the subject is a domestic animal. According to the preferred embodiment, the subject is a canine, a feline, an equidae, a ruminant or a porcine animal, for example, a dog, a horse, a cat, cattle, a pig, a sheep, a goat, a camel, etc. Most preferably, the subject is a dog.

The object of the present invention further relates a process of the preparation of MSCs for the treatment of osteoarthritis in a subject, which comprises the steps of:
a) isolating stromal vascular fraction (SVF) from at least two healthy donors that are different from the subject to be treated but belong to the same species;
b) providing MSCs obtained from each isolated fraction separately, preferably by digestion with collagenase;
c) sedimenting the obtained MSCs separately and resuspending them preferably in balanced saline buffer, for example Hank's buffer;
d) propagating the resuspended MSCs separately, preferably at least until 70%, 80%, or 90% confluency, then freezing them separately to generate a P₁ cell bank in a way that selecting and keeping those propagated MSCs prior freezing that are verified pathogen free, morphologically healthy, and based on the analysis of their cell surface markers, they are CD105, CD73 positive but CD45, CD34, CD14, CD11b, CD31, CD79α and CD19 negative, and their PD value during culturing is preferably in the range of 0.5 to 5, more preferably in the range of 1 to 4, most preferably about 3;
e) thawing part of a selected P₁ cell bank and further propagating MSCs to at least until 70%, 80%, or 90% confluency, then freezing them separately to generate a P2 cell bank in a way that selecting and keeping those propagated MSCs prior freezing that are verified pathogen free, morphologically healthy, and based on the analysis of their cell surface markers, they are CD105, CD73 positive but CD45, CD34, CD14, CD11b, CD31, CD79α and CD19 negative and their PD value during culturing is preferably in the range of 0.5 to 5, more preferably in the range of 1 to 4, most preferably about 3, and the level of kynurenine metabolite production of the MSCs is in the range of 30 to 50 ng/ml, preferably about 30 ng/ml, and the level of THBS1 cytokine production of the MSCs is in the range of 50 to 80 ng/ml, preferably about 50 ng/ml; and
f) repeating step e) until the number of MSCs reaches the amount of 10⁵ to 10⁸, preferably 10⁵ to 5×10⁷ separately, and the CPD value of the MSCs is preferably in the range of 3 to 14, more preferably in the range of 2 to 12, most preferably about 8.

Preferably, the MSCs are suspended in Hank's buffer containing 5% DMSO and are stored frozen.

Preferably, the MSCs are derived from at least two separate, preferably 3 to 15, more preferably 4 to 8, most preferably 5 to 7 donors that belong to the same species but are different from the individual to be treated.

Preferably, the MSCs are derived from stromal vascular fraction (SVF) which were isolated from abdominal adipose tissue removed during routine ovariohysterectomy.

The object of the present invention further relates to a process for the production of a composition for the treatment of osteoarthritis in a subject, which comprises the steps below:
a) isolating stromal vascular fraction (SVF) from at least two healthy donors that are different from the subject to be treated but belong to the same species;
b) providing MSCs obtained from each isolated fraction separately, preferably by digestion with collagenase;
c) sedimenting the obtained MSCs separately and resuspending them preferably in balanced saline buffer, for example Hank's buffer;
d) propagating the resuspended MSCs separately, preferably at least until 70%, 80%, or 90% confluency, then freezing them separately to generate a P₁ cell bank in a way that selecting and keeping those cultured MSCs prior freezing that are verified pathogen free, morphologically healthy, and based on the analysis of their cell surface markers, they are CD105, CD73 positive but CD45, CD34, CD14, CD11b, CD31, CD79α and CD19 negative and their PD value during culturing is preferably in the range of 0.5 to 5, more preferably in the range of 1 to 4, most preferably about 3;
e) thawing a part of a selected P₁ cell bank and further propagating MSCs to at least until 70%, 80%, or 90% confluency, then freezing them separately to generate a P₂ cell bank in a way that selecting and keeping those cultured MSCs prior freezing that are verified pathogen free, morphologically healthy, and based on the analysis of their cell surface markers, they are CD105, CD73 positive but CD45, CD34, CD14, CD11b, CD31, CD79α and CD19 negative and their PD value during culturing is preferably in the range of 0.5 to 5, more preferably in the range of 1 to 4, most preferably about 3, and the level of kynurenine metabolite production of the MSCs is in the range of 30 to 50 ng/ml, preferably about 30 ng/ml, and the level of THBS1 cytokine production of the MSCs is in the range of 50 to 80 ng/ml, preferably about 50 ng/ml;
f) repeating step c) until the number of MSCs reaches the amount of 10⁵ to 10⁸, preferably 10⁵ to 5×10⁷ separately, and the CPD value of the MSCs is preferably in the range of 3 to 14, more preferably in the range of 2 to 12, most preferably about 8;
g) mixing the MSCs thus selected and propagated from each donor separately depending on their level of kynurenine and THBS1 cytokine production in a way that we dispense fewer cells into the composition from those isolated MSCs that produce more kynurenine and THBS1 cytokine and more from those MSCs that produce less kynurenine and THBS1 cytokine and
h) adding one or more pharmaceutically acceptable additives to the balanced composition.

Preferably, the balanced composition is suspended in Hank's buffer containing 5% DMSO and is stored frozen.

As used herein, the term “Stem cells” refers to undifferentiated or not fully differentiated cells that are, on the one hand, capable of performing—in some cases a limited number of—divisions for self-maintenance and, at the same time, can generate one or more differentiated cell types.

The term “tissue stem cells” refers to multipotent stem cells that have at least partially remained undifferentiated during ontogenesis—preferably until adulthood—and are capable of differentiating into preferably a limited number or type of tissues, preferably into cell types characteristic of tissues or cells that are in contact with them. The role of tissue stem cells is to ensure the renewal of the tissues of the organism and the replacement of dead or aging cells.

The term “mesenchymal stem cells” (MSCs), as used herein, refers to multipotent—preferably stromal, that is of supportive tissue origin and/or localization—tissue stem cells that are i) adherent, that is capable of attaching to a surface, ii) capable of differentiating into at least bone, cartilage and adipose cells, and iii) CD105, CD73 positive but do not carry surface markers characteristic of hematopoietic stem and progenitor cells, and preferably do not carry surface markers characteristic of blood cell development lineages, that is, the MSCs are preferably CD45, CD34, CD14, CD11b, CD31, CD79α, and CD19 negative.

The term “morphologically healthy” refers to young, viable, smaller, fibroblast-like cells which are substantially free of senescent cells that are morphologically different from young cells: they are larger, flattened. Senescent cells are older, their functioning is altered, and do not only function improperly, but also damage the functions of cells in their surroundings.

As used herein, the term “pathogen free” refers to the propagated cell culture that does not contain any living pathogens characteristic of the given species that could cause infection when the produced end product is administered. The detection of pathogens is performed using pathogen-specific primers via PCR analysis, which detects even the lowest amounts of pathogens with high efficacy.

As used herein, the term “cumulative population doubling value, (CPD)” refers to the sum of the population doubling (PD) values calculated during culturing.

Population doubling value (PD) is determined with the following formula based on the initial cell number (X0) and the cell number determined at the end of culturing (N): PD=3.32 (log(N)−log(X0)). The PD value of MSCs is preferably in the range of 0.5 to 5, more preferably in the range of 1 to 4, most preferably about 3.

As used herein, the term “balanced composition” refers to a preparation of MSCs that are derived from at least two subjects of the same species, the MSCs isolated and selected from the separate donors are mixed in a kynurenine and THBS1 cytokine production-dependent way: fewer cells are dispensed into the end product from those isolated MSCs that produce more kynurenine and THBS1 cytokine and more from those MSCs that produce less kynurenine and THBS1 cytokine, thus, we obtain mixtures of MSCs with similar total kynurenine and total THBS1 cytokine production in the different amplification cycles (that is, in the MSC mixtures obtained later from other donors), which ensures that the preparations produced from independent cultures will have similar characteristics regarding cartilage regeneration.

As used herein, the term “stroma” or “supportive tissue” refers to a tissue that associates with or surrounds a given structure of the organism, e.g., an organ or preferably its functional tissue (“parenchyma”) and has a role in mechanically supporting the structure or organ or preferably the parenchyma. Preferably, the stroma contains or mostly contains or consists of conjunctive tissue. Preferably, the stroma is comprised of or mostly comprised of or consists of conjunctive tissue.

The “Conjunctive tissue” is a fibrous tissue that contains at least extracellular fibres, amorphous intercellular material (ground substance), and fixed and mobile cells where the fibres are embedded in the ground substance. Conjunctive tissues are, among others, the bone tissue, the tendon tissue, the cartilage tissue, and the adipose tissue. The conjunctive tissue supports and connects the internal organs, participates in bone formation, forms the walls of blood vessels, connects muscles to bones, and in case of injury replaces other types of tissues.

As used herein, the term “adipose tissue” refers to an animal conjunctive tissue that consists mainly of adipose cells (adipocytes) of mesenchymal origin, whose most important functions are energy storage, the protection of the organism from physical effects (cold, mechanical impact), and the cushioning of certain organs. Two types of adipose tissue can be distinguished: the yellow (also known as white) adipose tissue (which plays an important role in energy storage) and the brown adipose tissue (whose primary function is to generate body heat).

As used herein, the term “adipose cells” or “adipocytes” refers to cells that are specialized for the storage of energy in the form of fat in lipid vacuoles. Besides the presence of lipid vacuoles, adipocytes are preferably characterized by, among others, the expression of the following markers: PPARg, aP2 and HSL, Adipoq, Cebpa. Adipocytes can be white adipocytes characterized by, for example, their flat and peripheral nucleus, large vacuoles, and the secretion of adiponectin, rezistin, and leptin or can be brown adipocytes that are, for example, polygonal.

As used herein, the term “visceral” organs refers to the internal organs of the organism, particularly to those that are located in the abdominal cavity and the chest (thoracic cavity), e.g., the respiratory system, the digestive system, the urinary system, the internal genital organs, the liver, the heart, or the stomach.

“Visceral adipose tissue” is the adipose tissue associated with or in some cases surrounding the “visceral” organs.

As used herein, the term “subject” refers to a given animal being. Preferably, as used in the description, animal does not include human beings. Preferably, the animal is selected from mammals.

Kynurenic acid (KYNA), whose precursor is “kynurenine” (KYN), is a metabolite of tryptophane.

Thrombospondin 1, abbreviated as THBS1, is a protein encoded by the THBS1 gene. This protein is an adhesive glycoprotein that mediates cell-cell and cell-matrix interactions. This protein can bind to fibrinogen, fibronectin, laminin, collagen types V and VII and integrins alpha-V/beta-1. It plays an important role in platelet aggregation and tumorigenesis.

In the appendices of the application, unless indicated otherwise, the singular form of a word—regarding its meaning—encompasses the plural form. Analogously, the indefinite article “a” in front of a noun, unless indicated otherwise, encompasses the plural of the noun.

In the description, the term “comprises” should be understood in a way that the thing that “comprises” something may contain other elements besides the elements listed, but it contains—by all means—the listed elements. The term “comprises” encompasses the expression “essentially consists of” because this latter refers to the presence of other elements besides the ones listed, but these other elements do not contribute to the technical effect of the invention. In case of the term “consists of”, the thing that consists of something cannot contain other elements besides the ones listed, unless these are trivial elements to be implicitly included in the content of the thing that consists of something. The expression “comprises” can be limited to either of the expressions “essentially consists of” and “consists of”.

FIG. 1

1A shows the kynurenine metabolite production of the propagated MSCs per donation as measured via ELISA.

1B depicts the experiment in which we added 30 ng/ml kynurenine to the differentiating MSCs to examine its effect on the markers of hypertrophic chondrocytes. In the experiment, we measured the expression of the two main regulatory transcription factors of hypertrophic chondrocyte differentiation, factors GADD45 and CEBPb, and of their target, matrix metalloproteinase-13 (MMP13), by measuring their relative RNA levels on days 10 (early) and 24 (mature) of chondrogenic differentiation.

1C displays the results of this measurement.

FIG. 2

2A shows the THBS1 cytokine production of the propagated MSCs per donation as measured via ELISA.

2B depicts the experiment in which we added 50 ng/ml THBS1 to the differentiating MSCs to examine its effect on the markers of hypertrophic chondrocytes. In the experiment, we measured the expression of the two main regulatory transcription factors of hypertrophic chondrocyte differentiation, factors GADD45 and CEBPb, and of their target, matrix metalloproteinase-13 (MMP13), by measuring their relative RNA levels on days 10 (early) and 24 (mature) of chondrogenic differentiation.

2C displays the results of this measurement.

FIG. 3

3A shows the changes in the level of lameness following cartilage injury caused by the surgery and then MSC implantations with different doses (cell number). The figure clearly reveals that the balanced preparation gives the best results regarding improvement in lameness when applied at a dose of 2×10⁶.

3B, 3C, 3D demonstrate well how the MSCs selected according to the invention regenerate injured cartilage tissue compared to placebo.

3E displays how following the administration of the balanced MSC preparation produced according to the invention, new cartilage tissue develops over the damaged cartilage tissue.

FIG. 4 shows the effect of the balanced preparation produced according to the invention on the changing of lameness in the case of dogs (data were taken from the experiment described in FIG. 3 regarding placebo vs. target dose).

### DESCRIPTION OF EMBODIMENTS

Joints are formed at the intersection of bone ends. The movement of the bones is enabled by the layer of cartilage covering the bone surface and the synovial fluid (synovia) filling the joint cavity.

In case of osteoarthritis, the cartilage begins to wear to deeper and deeper layers, until the bone surface is uncovered, impeding friction-free movement of the bones. Small pieces may get detached from the surface of the cartilage and the bones, which—by moving in the joint cavity—can cause further inflammation and pain. The preparation of the synovial fluid also changes, and inflammatory cells appear in the neighbouring vessels.

Most of the owners would expect the animal to whine or whimper, unambiguously indicating pain in case of severe joint inflammation. However, this hardly ever occurs; the owners generally only notice that the animal is less active, moves more slowly, and is not happy to run for its favourite ball. Later on, it may be difficult for the animal to get up, jump off the couch, or go down the stairs, or it may be limping in one of its legs. These are all symptoms of chronic pain, which can worsen due to vigorous activity, after a long period of inactivity, or in cold weather. At the beginning, pain affects the limb only during movement, in advanced stages even in a resting position.

According to the present invention, the subject suffering from osteoarthritis is treated with a mixture of allogeneic stem cells derived from the adipose tissue of several healthy donors. The mixed stem cells derived from adipose tissue are not rejected by the treated animals.

According to the present invention, allogeneic MSCs are obtained from visceral adipose tissue employing a humane procedure or from surgical waste; thus, we collect them from visceral adipose tissue generated as surgical waste during the ovariectomy of healthy animals.

According to the present invention, a process for the production of a composition for the treatment of a subject suffering from osteoarthritis is disclosed, wherein said process comprises the separate isolation of mesenchymal stem cells from more than one, preferably 3 to 15, more preferably 4 to 8, most preferably 5 to 7 donor individuals that are different from the subject to be treated and diagnosed to be free of dysplasia and preferably of known diseases; the generation of a cell bank by freezing following a growth cycle; the testing for pathogens and, following this, two growth cycles or, in other words, passages; the determination of the cytokine profile of the propagated cells and, after the passages, the mixing of the fractions of the propagated mesenchymal stem cells (MSC) derived from the donors, in different ratios depending on the production of the kynurenine metabolite and the THBS1 cytokine; and the dispensing in a medium that ensures the survival of the cells during long-term storage by freezing and at the same time enables the direct injection of the preparation into the joint affected by osteoarthritis, following thawing and dilution.

In addition to the mesenchymal stem cells, the composition produced according to the process also contains DMSO as a cryoprotective agent and Hank's balanced salt solution (HBSS).

The preparation can be stored for a long period (more than 12 months) in liquid nitrogen. The composition is transported to the place of use in dry ice, and short storage (preferably 0 to 21 days, more preferably 1 to 14 days, and most preferably 2 to 7 days) at −80° C. is recommended, under which conditions the cells retain their viability and role in cartilage regeneration.

We isolated stromal vascular fraction (SVF) from abdominal adipose tissue removed during routine ovariohysterectomy from preferably 5 to 7 donors, which is cultured in vitro for at least 3 passages (P). Due to culturing, no other cells but the adipose tissue-derived mesenchymal stem cells survive, thus we obtained a pure MSC population of high cell numbers. As a result of culturing, no other cells apart from adipose tissue-derived mesenchymal stem cells (MSC) survive, thus, as a result of culturing we obtained a pure MSC population of high cell numbers. Cells were preferably released from the adipose tissue via digestion with a collagenase solution. We prepared a 0.1 to 1% collagenase solution in a “balanced salt solution”, that is, in a solution imitating physiological conditions (e.g., in Hank's buffer; the amount depends on the amount of adipose tissue to be treated). After isolation, cells were cultured in a medium suitable for the culturing of mammalian cells, e.g., Eagle's minimal essential medium (EMEM) or its Dulbecco-modified version, DMEM [Dulbecco's minimal essential medium (EMEM)], or preferably in F12-supplemented DMEM (DMEM/F12).

Further information about media suitable according to the present invention can be found, for example, in the review of Sato et al. (Sato, J. D. and Kan, M. 2001. Media for Culture of Mammalian Cells. Current Protocols in Cell Biology. (Published: October 1998)). Detailed guidance about the methods of culturing mammalian cells can be found in the publication of Phelan (Phelan, M. C. 2007. Basic Techniques in Mammalian Cell Tissue Culture. Current Protocols in Cell Biology. (Published: 1 Sep. 2007)) and Vinci V. A. et al. (Vinci, Victor A., Parekh, Sarad R. Eds.: Handbook of Industrial Cell Culture: Mammalian, Microbial, and Plant Cells; Published by Humana Press Inc. in 2003).

After SVF isolation and culturing to one passage (P1), a cell bank (P1 cell bank) was generated from the P1 cells in each case by freezing, and only those cells were propagated that we verify to be pathogen free, thus eliminating the risk of culturing cells that could be potential sources of pathogens and the transmission of infections from the donor individual to the treated animal. After thawing, pathogen-free P1 cell bank isolates were propagated for at least two further passages (P2 and P3).

Surprisingly, our experiments demonstrated that those propagated MSCs that have the kynurenine metabolite and THBS1 production in a certain range, can suppress the hypertrophic chondrocyte phenotype during differentiation as a result of the decrease in the expression of GADD45 and CEBPb and the MMP13 protein they target. Suppressing the development of the hypertrophic chondrocyte phenotype, in the end, enhanced the restoration of joint tissue in case of abnormal conditions. This composition provides a more efficient treatment than propagated MSC cell isolates that also comprised allogeneic MSCs but ones that did not produce kynurenine and THBS1 at appropriate levels.

To dispense cells of appropriate condition, the production of certain cytokines, e.g., THBS1, cell morphology, duplication time, MSC surface markers, karyotype, and kynurenine production in the P2 or P3 stages were tested. Five to seven isolates from the culture vessel were obtained, and depending on their level of kynurenine metabolite and THBS1 cytokine production, the cells were mixed in a way that fewer cells were dispensed into the final balanced composition from those isolated MSCs that produce more kynurenine and THBS1 cytokine and more from those MSCs that produce less kynurenine and THBS1 cytokine, thus, mixtures of MSCs with similar total kynurenine and total THBS1 cytokine production in the different amplification cycles were obtained (that is, in the MSC mixtures obtained later from other donors), which ensured that the preparations produced from independent cultures have similar characteristics regarding cartilage regeneration. The cell mixture was washed with Hank's buffer. The cell pellet was suspended in DMSO-containing Hank's buffer, dispensed into freezing vials, and stored in liquid nitrogen. A single dose contained 500.000-10.000.000 cells; this is what we call a balanced composition. We measured the endotoxin levels in the end product.

The composition could be transported to the place of use in dry ice. The cells could be stored for a short period (preferably 0 to 21 days, more preferably 1 to 14 days, and most preferably 2 to 7 days) in dry ice or frozen at −80° C. The freezing medium ensures that the cell preparation retains its viability even during long-term storage and, furthermore, enables direct intraarticular injection after thawing, without having to remove it from the cells at the place of use (which could not even be performed in an average veterinarian's office).

The balanced composition contained MSCs that have passed quality control tests, were derived from preferably five to seven types of donors and were mixed in a ratio depending on their kynurenine and THBS1 production.

Dogs suffering from osteoarthritis put less load on the affected leg due to joint pain, which manifests as lameness during walk. The primary testing of the preparation was performed by veterinarians; during further in vivo experiments, the condition of the treated animal was evaluated by the owner in a standardized questionnaire; this was supplemented with a standardized questionnaire filled in by the veterinarian. As a result of the measurements, it was statable that the balanced composition can be used to treat osteoarthritis.

### EXAMPLES

### 1. Donor Selection:

Small or medium-sized mixed-breed female dogs that had received their compulsory vaccinations (against rabies) served as adipose tissue donors. We determined a wider upper age limit of 2 to 8 years, a stricter one of 2 to 6 years, and an optimum of 3 years. Adipose tissue was obtained from the waste product of routine ovariohysterectomy; thus, invasive surgery is not needed for the manufacturing of mesenchymal stem cells (MSCs). The MSC isolates were prepared from each donor separately. Adipose tissue was transported in a sterile container in 25 ml Hank's balanced salt solution (HBSS), cooled (4-10° C.) to the place of processing, culturing.

### 2. MSC Isolation, Propagation, Generation of the P1 Cell Bank

a. Isolation
i. Preparation of the tissue for digestion
ii. It was digested in 0.2% collagenase-containing HBSS solution. The digestion was blocked with 5% FCS and then washed. After washing, the cell pellet was resuspended in DMEM/F12+10% FCS solution (culture medium) in a way that there was 1 g of adipose tissue in 3 ml culture medium.

With two independent samplings from the cell pellet: 50 μl cells+450 μl DT solution (800 μl 0.2% trypane blue solution+200 μl distilled water) we counted the cells based on the average.

From preferably 5 to 9 g, more preferably 6 to 8 g of adipose tissue, preferably 5×10⁶ to 20×10⁶, more preferably 7×10⁶ to 15×10⁶, optimally 14×10⁶ cells can be isolated.

b. Propagation of Isolated Cells
i. We cultured the isolated cells in a cell culture incubator until they reached 70-80% confluency. This value was achieved preferably in 1 to 10, more preferably in 2 to 8, most preferably in 5 days depending on the donor. The complete medium used during propagation:
   DMEM/F12 (Dulbecco's Modified Eagle Medium/Nutrient Mixture F-12) medium
   10% FCS (foetal calf scrum)
   50 μg/ml Penicillin/Streptomycin
      c. Collection of P1 Cells

Culture medium was removed from the P1 cell culture, and the cells were collected in centrifuge tubes. We took a sample from the thus obtained cell suspension and perform pathogen analysis to detect donor-derived pathogens.

d. Freezing a P1 Cell Bank

The afore-obtained cell suspension was centrifuged, the cell pellet was resuspended in FCS containing 10% DMSO, distributed into freezing vials, and stored for a maximum of 24 hours at −80° C. and for long-term storage in liquid nitrogen. Herewith, we have created the P1cell bank.

e. Quality Control of P1 Cells
i. Calculation of Population Doubling

The population doubling value (PD) was determined by the following formula based on the initial cell number (X0) and the cell number determined at the end of P1 culture (N): PD=3.32 (log N−log X0).

The PD value of the MSCs was preferably in the range of 0.5 to 5, more preferably in the range of 1 to 4, most preferably about 3.

ii. Testing Cell Morphology

Before collection, the propagated cells were analysed under a microscope and documented. We performed this analysis before every passage since morphology provides feedback on the condition of the cells. We examined whether senescent cells appear in the culture during propagation; they were different in shape: larger, flatter. (Senescent cells are older, their functioning is altered, and do not only function improperly, but also damage the functions of cells in their surroundings.)

iii. Detection of Donor-Derived Pathogens in P1 Cells

We prepared a dilution series from the nucleic acids of 26 known canine pathogens (Table I) in MSC lysate and determined the limit of detection for the pathogens, that is, the sensitivity of the PCR-based (polymerase chain reaction) test we employed. This proceeds as follows:

We isolated nucleic acid from the MSC lysate.

First, we determined the sensitivity of the PCR-based (polymerase chain reaction) test we employed, for which we isolated nucleic acid from the 26 known canine pathogens (that is, DNA viruses, such as the Adenoviridae, for example, the Canine adenovirus type-1 (CAV-1), such as the Parvoviridae, for example, Canine parvovirus (CPV), such as the Herpesviridae, for example, Canine herpesvirus (CaHV-1) Canid herpesvirus 1, Canid alphaherpesvirus 1, Swine herpesvirus 1 (pseudorabies viruses), Suid herpesvirus 1 (SuHV1), Suid alphaherpesvirus 1, such as Papillomaviridae, for example, caninc oral papillomavirus (COPV), ncbi: Lambdapapillomavirus 2; RNA viruses such as Paramyxoviridae, for example, distemper morbillivirus (CDV), canine parainfluenza virus (CPIV), such the Orthomyxoviridae, for example, the canine influenza virus (CIV), such as Rhabdoviridae, for example, rabies virus (Rabies lyssavirus), such as Coronaviridae, for example, canine coronavirus CCOV (Type I, and II); bacteria such as Mycoplasmataceae, for example, Mycoplasma, such as Leptospiraceae, for example, Leptorpira interrogans serovars: serovars Icterohaemorrhagiae, Copenhagi, Canicola, such as Sporichactaceae, for example, Borrelia, such as Brucellaceac, for example, Brucella canis, such as Anaplasmataceac, for example, Ehrlichia canis, Anaplasma phagocytophilum, Anaplasma platys, Neorickettsia, Rickettsia, such as Bartonellaceae, for example, Bartonella vinsonii/Bartonellosis; Protozoans, such as Phylum: Apicomplexa/Babesiidae, for example, Babesia, such as Phylum: Apicomplexa/Sarcocystidae, for example, Neospora caninum, such as Phylum: Euglenozoa/Trypanosomatidae, for example, Trypanosoma cruzi/Trypanosomiasis, such as Phylum: Euglenozoa/Trypanosomatidae, for example, Leishmania, such as Nematoda/Onchocercidae, for example, Dirofilaria immitis, intestinal roundworms (Toxocara canis, Toxascaris leonina, Ascaris lumbricoides)) and prepared a dilution series from the obtained nucleic acids in MSC lysate. To determine the detection limit, we performed PCR with pathogen-specific primers and defined the smallest nucleic acid concentration that still results in a PCR product. Next, PCR reactions with pathogen-specific primers were performed on samples from P1 cells derived from different donors. A positive signal indicated that the MSC culture is infected with that given pathogen. Those MSCs were propagated further that prove to be pathogen free in the PCR analysis.

### 3. Propagation of Pathogen-Free MSCs Until P3

a. P2 Propagation

After thawing, the cells were cultured in an incubator until the cell culture covered 90% of the bottom of the culture dish. The culture medium on the cells was changed every 3 to 4 days. At the end of the P2 culture phase, 15×10⁶ to 45×10⁶ cells obtained per donor. The PD value determined as described above was preferably in the range of 0.5 to 5, more preferably in the range of 1 to 4, most preferably about 2.

Cell morphology was also tested as described above.

b. P3 Propagation and Quality Control

Cells were cultured until the cell culture covered 80% of the bottom of the culture dish. The culture medium on the cells was changed every 3 to 4 days.

Cell morphology was tested.

The PD value determined as described above was preferably in the range of 0.5 to 5, more preferably in the range of 1 to 4, most preferably about 2.

We determined the cumulative population doubling value (CPD) which was identical to the sum of the population doubling values calculated during the propagation. We determined the cumulative population doubling value (CPD) which was identical to the sum of the population doubling values calculated during the propagation.

During propagation, the CPD value of the MSCs was preferably in the range of 3 to 14, more preferably in the range of 2 to 12, most preferably about 8.

We tested the purity of the population with cell surface markers:
they were CD105 and CD73 positive but did not carry surface markers characteristic of hematopoietic stem cells and progenitor cells and preferably did not carry surface markers characteristic of blood cell development lineages, that is, the MSCs were preferably CD45, CD34, CD14, CD11b, CD31, CD79α, and CD19 negative.

We tested whether the MSC isolates can be differentiated into chondrocyte, osteoblast and adipocyte directions.

We determined the cytokine profile of the cells, for which we took a sample from the supernatant of the P3 cells. ELISA (Enzyme linked immunosorbent assay) method was used to examine the produced amounts of the following cytokines: Thrombospondin-1 (THBS1), Fibroblast Growth Factor 1 (FGF1), Fibroblast Growth Factor 2 (FGF2), Hepatocyte growth factor (HGF), Stem Cell Factor (SCF), Transforming Growth Factor β (TGFβ), Interleukin 1 receptor antagonist (ILIRa), Interleukin 10 (IL-10), and Interleukin-13 (IL-13). The measurements were made using MSCs derived from 5 to 7 donors during two-three independent propagations; we determined which cytokines were produced by the MSCs under our manufacturing conditions and set the upper and lower limits of the amounts of cytokines produced. Further on, these values were tested periodically during production, and those MSC isolates was used that produce the cytokines selected preferably from the above-mentioned ones within the range we set, that is, this value provided feedback that we produced MSCs of the same quality under the given manufacturing conditions.

We measured the kynurenine and THBS1 production of the cells and, based on that, we determined the ratio of the MSC isolates comprising the preparation in the final distribution. In order to do this, we took a sample from the culture medium on the cells and, after adding Ehrlich reagent, the colour change of the reaction mix were monitored. ELISA methods can also be used for the measurement. The measured values were normalized to the cell number after collecting the cells, and we added less from those MSCs that produce more kynurenine and THBS1 cytokine and more from those that produce less to the mix.

The final balanced MSC composition is characterized by the following:
a. allogeneic (not its own),
b. derived from at least two healthy donors that are different from the subject to be treated;
c. morphologically healthy, substantially free of senescent cells;
d. free of pathogens;
e. possesses the main characteristics of MSCs; they are CD105 and CD73 positive but do not carry surface markers characteristic of hematopoietic stem cells and progenitors and preferably do not carry surface markers characteristic of blood cell development lineages; that is, the MSCs are preferably CD45, CD34, CD14, CD11b, CD31, CD79α and CD19-negative;
f. during propagation, the CPD value of the MSCs is preferably 3-14, more preferably 2-12, most preferably 8;
g. the level of kynurenine metabolite production of the MSCs is 30-50 ng/ml, preferably approximately 30 ng/ml, and the level of THBS1 cytokine production of the MSCs is 50-80 ng/ml, preferably approximately 50 ng/ml.
h. a mixture of MSC cell cultures depending on the level of kynurenine and THBS1 production, that is, the cells are mixed in a way that fewer cells are dispensed into the balanced preparation from those isolated MSCs that produce more kynurenine and THBS1 cytokine and more from those MSCs that produce less kynurenine and THBS1 cytokine,
i. number of cells 100.000-50.000.000
j. cell suspension in freezing medium (5% DMSO in Hank's (HBSS) solution).

Furthermore, the object of the invention relates to a composition containing MSCs to be used for the treatment of osteoarthritis in subject needs thereof.

The 5% DMSO-containing Hank's buffer in the end-product ensures that the cells retain their viability during long-term storage and, at the same time, following dilution, they can be injected into the joint directly, which is proven by in vivo results. The MSC balanced preparation was transported in dry ice and, at the place of use, if the product is not administered immediately, it can be stored at −80° C.

The balanced composition should be used preferably within 0 to 21 days, more preferably within 1 to 14 days, and most preferably within 2 to 7 days of storage at −80° C.

Dogs treated with the MSCs stored at −80° C. for 5 days tolerated the treatment well, and the composition proved to be efficacious.

The product was thawed immediately before use at 37° C., supplemented to the required amount with 37° ° C. PBS solution, and mixed. The cell suspension was drawn into a syringe with an injection needle and injected slowly into the affected joint.

### The Effect of Kynurenine and THBS1 on the Differentiating MSCs

Table 1 displays the results of the experiment in which following 7 days of differentiation, we added 30 ng/ml kynurenine (KYNA) or 50 ng/ml THBS1 cytokine to the differentiating MSCs for 5 days and, using next-generation sequencing, we examined the effects of these treatments on the differentiating chondrocytes. The transcriptional changes taking place as a result of the treatments were summarized via “GO analysis”. Our results show that the KYNA and the THBS1 treatments significantly affected cell division and several key processes related to cartilage formation. Based on these results, we examined the effect of the KYNA or THBS1 treatments on key genes that advantageously affect chondrocyte differentiation.

TABLE 1
The results of the transcriptome analysis

| | | THBS1 | KYNA |
|---|---|---|---|
| | | treatment | treatment |
| GO term ID | GO Term | (Significance) | (Significance) |
| GO:0000278 | mitotic cell cycle | 4.6E−08 | 3.45E−11 |
| GO:0001503 | bone formation | 1.72E−07 | 0.0002 |
| GO:0001568 | blood vessel formation | 1.46E−06 | 4.58E−06 |
| GO:0051216 | cartilage formation | 0.0042 | 0.023 |
| GO:0031012 | extracellular matrix | 1.45E−06 | 0.00089 |
| GO:0062023 | collagen-containing | 1.09E−05 | 0.018 |
| | extracellular matrix | | |

### The Efficacy of MSCs in Suppressing the Expression of Hypertrophic Genes During Chondrocyte Differentiation

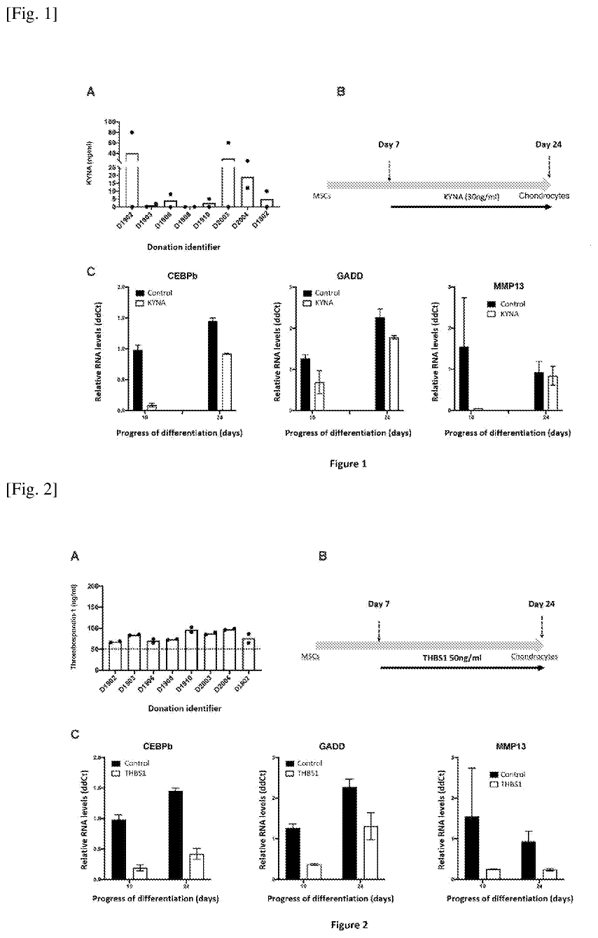

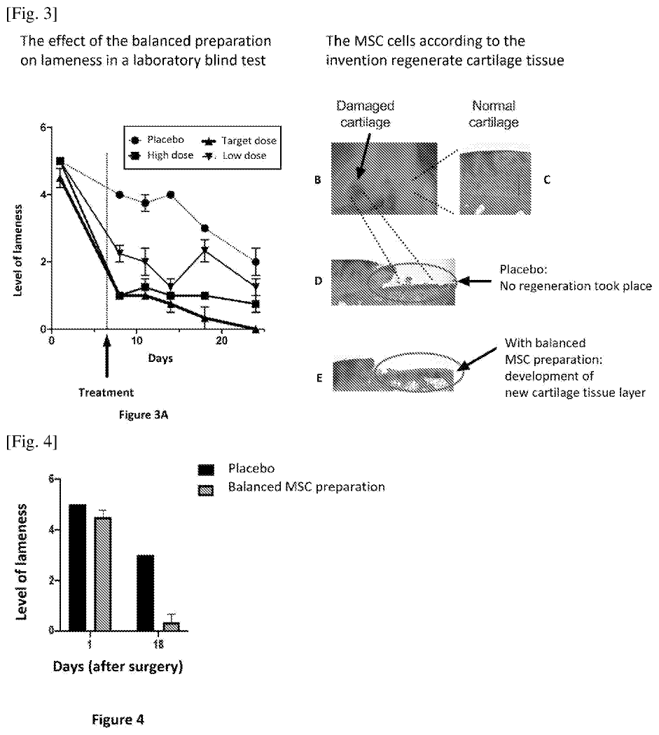

With the analysis, we demonstrate the efficacy of the selected specific MSCs in modifying chondrocyte differentiation towards a joint-regenerating phenotype. During the analysis, first we quantified the levels of KYNA produced in canine AD-MSCs derived from 8 independent donations by ELISA in passage 3 cultures. The results of this analysis are shown in [FIG. 1], demonstrating variable production of KYNA in the range of 0 to 80 ng/ml with 30 ng/ml differences between the propagations. In order to determine whether the level of KYNA produced by the MSCs is sufficient to promote chondrogenesis of canine MSCs and to evaluate its effect on markers of hypertrophic chondrocytes, we carried out an experiment ([FIG. 1]) in which we added 30 ng/ml KYNA to differentiating canine MSCs at day 7 and measured the expression of two main regulatory transcription factors of hypertrophic chondrocyte differentiation, GADD45 and CEBPb, as well as one of their targets, matrix metalloproteinase-13 (MMP13) by determining their relative RNA levels at days 10 (early phase) and 24 (mature phase) of chondrogenic differentiation. The results presented in [FIG. 1] demonstrate that 30 ng/ml KYNA is able to suppress the hypertrophic chondrocyte phenotype by reducing the expression of GADD45 and CEBPb and their target protein MMP13.

In another analysis, we quantified the levels of THBS1 produced in the AD-MSCs derived from 8 independent donations by ELISA in passage 3 cultures. Results of this analysis are shown in [FIG. 2], demonstrating variable production of THBS1, in the range above 50 ng/ml, with little difference between propagations. In order to test whether the level of THBS1 produced by the MSCs is sufficient to promote chondrogenesis of canine MSCs and to evaluate its effect on markers of hypertrophic chondrocytes, we carried out an experiment depicted in [FIG. 2]. During the analysis, we added 50 ng/ml THBS1 to differentiating canine MSCs at day 7 and measured the expression of two main regulatory transcription factors of hypertrophic chondrocyte differentiation, GADD45 and CEBPb, as well as one of their targets, matrix metalloproteinase-13 (MMP13) by determining their relative RNA levels at days 10 (early phase) and 24 (mature phase) of chondrogenic differentiation. The results presented in FIG. 2 demonstrate that 50 ng/ml THBS1 was sufficient to suppress the hypertrophic chondrocyte phenotype by reducing the expression of GADD45 and CEBPb and their target protein MMP13.

### The Regenerative Effect of MSCs in the Treatment of Induced Osteoarthritis in Dogs:

We proved the efficacy of the balanced composition containing AD-MSCs selected as described previously in the treatment of osteoarthritis in dogs, in several steps. First, we tested the regenerative effect of the balanced preparation on induced osteoarthritis under experimental conditions, then we monitored the healing of natural cartilage wear in owned dogs.

To examine the treatment of artificially induced osteoarthritis with the balanced composition, unilateral cartilage defect of 4 mm diameter affecting the entire width of the medial condyle of the knee joint was induced artificially on the first day. The transplantation was performed on the 7th day after surgery. One dog was injected with MSCs of the balanced preparation per group; the control animal was treated with the carrier of the MSCs. The animals received MSC treatment once, and they were monitored for the following three weeks, with the level of lameness recorded daily. During the study, we followed changes in body weight, food consumption, and haematological values.

In sum, Beagles tolerated the MSC treatment of the knee joint well. The examined dose proved to be efficacious in the treatment of artificially induced cartilage defect. The daily evaluation of the condition of the animals was performed by two people, independent of each other. The results of the evaluation are presented in [FIG. 3]. [FIG. 3] also displays the histological analysis. The treatment of artificially induced osteoarthritis in dogs with the balanced MSC preparation according to the present invention resulted in the differentiation of appropriate chondrocytes and the regeneration of new cartilage tissue at the site of injury.

Further on, the evaluation of the accepted veterinary symptoms is based on the observations of the owner and the veterinarian: lameness during walk and trot, pain to touch and pressure, range of motion, functional abilities, need for anti-inflammatory/pain-reducing medication, scoring of symptom-free period from 1 to 5, with which the level of lameness can be monitored. The presented method has several advantages. The main advantage of the method is that dogs suffering from osteoarthritis load their affected legs to a lesser extent, which manifests as lameness during walk, thus the change can be monitored easily. The primary testing of the balanced preparation was performed by veterinarians, and, during further in vivo tests, the conditions of the treated dogs were evaluated by the owners based on standard questionnaires. This method gives a relative value indicating how the loading of the affected leg changes during walk as a result of the treatment, due to the decreased joint pain resulting from cartilage regeneration, and the loading of the affected leg increases as a result of decreased pain.

Besides the examples above, the present invention can be realised through other steps within the scope of protection.

### The Regenerative Effect of MSCs in the Treatment of Osteoarthritis in Horses:

We proved the efficacy of the balanced preparation containing AD-MSCs selected according to the invention in the treatment of osteoarthritis in horses, with the procedures described previously, by monitoring the healing of natural cartilage wear in horses.

For the transplantation, one horse was injected intraarticularly with MSCs of the balanced preparation per group; the control individual was treated with the carrier of the MSCs. The animals received MSC treatment once, and after that, they were monitored for the following three weeks, and the level of lameness was recorded daily.

In sum, the horses tolerated well the treatment of the knee joint with MSCs according to the invention. The examined dose proved to be efficacious in the treatment of lameness. The daily evaluation of the condition of the animals was performed by two people, independent of each other.

Further on, the evaluation of the accepted veterinary symptoms is based on the observations of the owner and the veterinarian: lameness during walk and trot, pain to touch and pressure, range of motion, functional abilities, need for anti-inflammatory/pain-reducing medication, scoring of symptom-free period from 1 to 5, with which the level of lameness can be monitored. The presented procedure has several advantages. The main advantage of the method is that horses suffering from osteoarthritis load their affected legs to a lesser extent, which manifests as lameness during walk, thus the change can be monitored easily. The primary testing of the balanced preparation was performed by veterinarians, and, during further in vivo tests, the conditions of the treated horses were evaluated by the owners based on questionnaires. This latter method gives a relative value indicating how the loading of the affected leg changes during walk as a result of the treatment, due to the decreased joint pain resulting from cartilage regeneration, and the loading of the affected leg increases as a result of decreased pain. The results demonstrated that treatment with the balanced MSC preparation according to the invention resulted in the regeneration of cartilage tissue at the site of injured cartilage, and following treatment, lameness improved significantly, ceased.

### COMPARATIVE ANALYSIS

Comparing the composition according to present invention to other MSC compositions according to the prior art, it can be stated that former is more efficacious in the treatment of osteoarthritis. The amount of MSCs that are revealed in previous publications is in all cases an order of magnitude higher. And their efficacy based on studies of lameness is lower than that of the composition according to present invention (e.g., Kriston-Pal et al. 2017). While in the case of the composition according to the present invention the regeneration of cartilage tissue was obviously detectable, such level of regeneration was not reported in previous documents. In the studies we performed on dogs by the administration of compositions according to the prior art, the degree of lameness hardly improved, while in the case of the composition according to the present invention the degree of lameness improved by several orders of magnitude, in most of the cases the dog's tread was restored after treatment.

1. Cell culture of mesenchymal stem cells (MSC) characterized in that:
a. they are derived from at least two healthy allogeneic donors;
b. they are morphologically healthy, substantially free of senescent cells;
c. they are free of pathogens characteristic of the species;
d. they possess the main characteristics of MSCs, that is, they are CD 105 and CD73 positive but do not carry surface markers characteristic of hematopoietic blood stem cells and progenitor cells and preferably do not carry surface markers characteristic of blood cell lineages, that is, the MSCs are preferably CD45, CD34, CD14, CD11b, CD31, CD79α, and CD19 negative;
e. during propagation, the cumulative population doubling value, that is, CPD value of the said MSCs is preferably in the range of 3 to 14, more preferably in the range of 2 to 12, most preferably about 8;
f. the level of kynurenine metabolite production of the MSCs is in the range 30 to 50 ng/ml, preferably about 30 ng/ml, and the level of THBS1 cytokine production of the MSCs is in the range of 50 to 80 ng/ml, preferably about 50 ng/ml;
g. number of cells is in the range of 10⁵ to 5×10⁷.

2. The MSC culture according to claim 1 characterised in that the MSC culture is a mixture of MSCs selected from the separately propagated donations, wherein the mixture contains isolated MSCs producing kynurenine at about 50 ng/ml and THBS1 cytokine at about 80 ng/ml in an amount less than 50% of the mixture and isolated MSCs producing kynurenine at about 30 ng/ml and THBS1 cytokine at about 50 ng/ml in an amount of higher then 50% of the mixture.

3. The MSC culture according to claim 1 characterised in that the said subject is a canine, a feline, an equidae, a ruminant or a porcine animal, preferably a dog, a horse, a cat, cattle, a pig, a sheep, a goat, and most preferably a dog or a horse.

4. The MSC culture according to claim 1 characterised in that the cell culture is a suspension and further comprises a freezing medium, preferably 5% DMSO in Hank's buffer solution.

5. A balanced MSC composition which comprises the MSC culture according to claim 2, and one or more pharmaceutically acceptable additives.

6. A process for the production of a composition for the treatment of a subject suffering from osteoarthritis characterised in that it comprises the following:
a) isolating stromal vascular fraction (SVF) from the adipose tissue of at least two healthy donors that are different from the subject to be treated but belong to the same species;
b) providing the mesenchymal stem cells, that is, the MSCs obtained from each isolated fraction separately, preferably by digestion with collagenase;
c) sedimenting the obtained MSCs separately and resuspending preferably in balanced saline buffer, for example Hank's buffer;
d) propagating the resuspended MSCs separately, preferably at least until 70%, 80%, or 90% confluency, then freezing them separately to generate a P1 cell bank in a way that selecting and keeping those propagated MSCs prior freezing that are verified pathogen free, morphologically healthy, and based on the analysis of their cell surface markers, they are CD 105, CD73 positive but do not carry surface markers characteristic of hematopoietic stem cells and progenitor cells and preferably do not carry surface markers characteristic of blood cell development lineages, that is, the MSCs are preferably CD45, CD34, CD14, CD11b, CD31, CD79α, and CD19 negative, and their PD value during culturing is preferably in the range of 0.5 to 5, more preferably in the range of 1 to 4, most preferably about 3;
e) thawing a part of the separate P₁ cell banks and further propagating MSCs to at least until 70%, 80%, or 90% confluency, then freezing them separately to generate a P₂cell bank in a way that selecting and keeping those cultured MSCs prior freezing that are verified pathogen free, morphologically healthy, and based on the analysis of their cell surface markers, they are CD105, CD73 positive but do not carry surface markers characteristic of hematopoietic stem cells and progenitor cells and preferably do not carry surface markers characteristic of blood cell development lineages, that is, the MSCs are preferably CD45, CD34, CD14, CD11b, CD31, CD79α and CD19 negative, and their PD value during culturing is preferably in the range of 0.5 to 5, more preferably in the range of 1 to 4, and most preferably about 3, and the level of kynurenine metabolite production of the MSCs is in the range of 30 to 50 ng/ml, preferably about 30 ng/ml, and the level of THBS1 cytokine production of the MSCs is in the range of 50 to 80 ng/ml, preferably about 50 ng/ml;
f) repeating step e) until the number of MSCs reaches the value of 10⁵ to 10⁸, preferably 10⁵ to 5×10⁷ separately, and the CPD value of the MSCs is preferably in the range of 3 to 14, more preferably in the range of 2 to 12, most preferably about 8;
g) optionally mixing the MSCs thus obtained, propagated from each donor separately, wherein the mixture contains isolated MSCs producing kynurenine at about 50 ng/ml and THBS1 cytokine at about 80 ng/ml in an amount less than 50% of the mixture and isolated MSCs producing kynurenine at about 30 ng/ml and THBS1 cytokine at about 50 ng/ml in an amount of higher then 50% of the mixture and;
h) adding 5% DMSO Hank's (HBSS) solution to the balanced composition.

7. The process according to claim 6 characterised in that MSCs are isolated from the adipose tissue of preferably 3 to 15, more preferably 4 to 8, and most preferably 5 to 7 separate healthy subjects.

8. The process according to claim 6 characterised in that the adipose tissue is a surgical waste product of ovariohysterectomy of a non-human subject.

9. The process according to claim 6 characterised in that the subject is a canine, a feline, an equidae, a ruminant or a porcine animal, preferably a dog, a horse, a cat, cattle, a pig, a sheep, a goat, a camel, more preferably a dog or a horse.

10. The balanced composition according to claim 5, characterised in that the balanced composition is used for the treatment of a subject suffering from osteoarthritis.
